(19) United States
(12) Patent Application Publication
(10) Pub. No.: US 2026/0168960 A1
(43) Pub. Date: Jun. 18, 2026

(54) MICROFLUIDICS-BASED HIGH CONCENTRATION DETECTION METHOD FOR LABEL-FREE TARGET

(71) Applicants: ProvaLabs, Inc., Seoul (KR); SEOUL NATIONAL UNIVERSITY R&DB FOUNDATION, Seoul (KR)

(72) Inventors: Sungjae HA, Seongnam-si, Gyeonggi-do (KR); Sung Jae KIM, Seoul (KR); Taewan KIM, Suwon-si, Gyeonggi-do (KR); Tae Hyun KIM, Seoul (KR); Sang Woo SEO, Seoul (KR)

(21) Appl. No.: 19/136,779

(22) PCT Filed: Dec. 12, 2023
(86) PCT No.: PCT/KR2023/020373

§ 371 Date: Jun. 6, 2025

(30) Foreign Application Priority Data
Dec. 13, 2022 (KR) 10-2022-0174175
Dec. 11, 2023 (KR) 10-2023-0178708

(51) Int. Cl. G01N 27/447; C12Q 1/6825

(52) U.S. Cl. CPC G01N 27/44791; C12Q 1/6825; G01N 27/44726

(57) ABSTRACT

Provided is a method for detecting a label-free target material using ion concentration polarization (ICP), the method including the steps of (a) injecting a sample solution containing a fluorescently labeled probe material and a label-free target material into a microchannel; (b) applying voltage to both ends of the microchannel; (c) acquiring an image of the concentrated state of the sample solution; and (d) calculating a separation index from the acquired image to determine the presence or absence of a target.

### TECHNICAL FIELD

The present invention relates to a micro/nanofluidics-based concentration detection method for a label-free target, and more particularly, to a selective preconcentration detection method for a label-free target using ion concentration polarization (ICP) in a micro/nanofluidic concentration control system.

This invention is the result of research supported by the Ministry of SMEs and Startups (MSS) under the ‘Industry-Academia Collaboration R&D’ program. [Project Title: Development of Optical Detection Method for Non-Labeled DNA Target by Nanoelectrokinetics and CRISPR/Cas Technology, Project Number: RS-2023-00223086, Project Identification Number: 1425175756]

### BACKGROUND ART

In the bio and environmental fields such as medical care, pharmaceuticals, drug testing, water quality testing, and clinical diagnostics, sample concentration has been studied as a critical technology for improving detection and analysis efficiency due to the extremely low concentrations of samples.

Current sample concentration methods in micro-scale systems include field-amplified sample stacking (FAS), isotachophoresis (ITP), electrokinetic trapping, micellar electrokinetic sweeping, chromatographic preconcentration, and membrane preconcentration. These techniques may enable concentration regardless of sample types and offer high concentration factors. However, because the concentration occurs inside microchannels, only very small amounts of samples (picoliters (pL) to nanoliters (nL)) are concentrated, resulting in low detection sensitivity. In addition, external control devices for driving concentration devices are required, and only skilled researchers can properly operate the concentration devices due to large variations in control parameters depending on samples and microfluidic devices.

Microsample concentration systems that use ion concentration polarization (ICP) for heavy metal detection in water testing, hospital diagnostics, protein purification, etc. have also been reported. However, because the concentration behavior depends on the electrochemical properties of the sample, the process of conducting concentration experiments and identifying stable concentration conditions often relies on the experience of skilled researchers. Therefore, the difficulty of broad use by non-experts serves as a barrier to the commercialization of technology.

A technique that uses the ICP-based separation and concentration phenomenon and the CRISPR/Cas genome-editing technology to detect the presence of a target substance in a sample based on the relative position between a target labeled with a specific fluorescent color and a ribonucleoprotein (RNP) complex labeled with another fluorescent color and specifically bound to the target has been proposed. However, this method is more cumbersome than label-free detection methods because fluorescent labeling of the target is required. Moreover, because the target and probe substances must be fluorescently labeled with different colors, a complex fluorescence optical system is required to distinguish between the labels.

### DETAILED DESCRIPTION OF THE INVENTION

### Technical Problem

The present invention provides a micro/nanofluidics-based concentration detection method for a label-free target, by which the label-free target may be detected using only the fluorescence signal of a probe substance for detection. However, the above description is an example, and the scope of the present invention is not limited thereto.

### Technical Solution

According to an aspect of the present invention, there is provided a method of detecting a label-free target substance using ion concentration polarization (ICP).

The method may include (a) injecting a sample solution including a fluorescently labeled probe substance and a label-free target substance, into a microchannel, (b) applying a voltage to each end of the microchannel, (c) obtaining an image showing a concentration status of the sample solution, and (d) determining presence of the target by calculating a separation index from the obtained image.

In step (b), an ion depletion zone and a preconcentration plug may be formed.

In step (d), a single preconcentration plug may be separated into two plugs, and one of the two plugs may be a preconcentration plug of a free labeled substance (hereinafter referred to as a first preconcentration plug), while the other may be a concentration plug of a labeled substance bound to the label-free target substance (hereinafter referred to as a second preconcentration plug).

The first preconcentration plug may exhibit concentration with a concentration equilibrium point that moves, while the second preconcentration plug may exhibit concentration at a point in the channel or with a concentration equilibrium point that moves more slowly than the first preconcentration plug.

The separation index may be calculated by Equation 1.

$$Separation index ( S ) = \frac{{Δ}_{P - P}}{( {FWHM}_{1} + {FWHM}_{2} ) / 2} \qquad ( Equation 1 )$$

(where Δ_{P-P} denotes a distance between centers of the first and second preconcentration plugs, FWHM₁ denotes an effective width of the first preconcentration plug, and FWHM₂ denotes an effective width of the second preconcentration plug.)

When the separation index is lower than a target threshold in step (d), (e) adjusting electrical or mechanical control conditions to be applied to the microchannel may be performed least once after step (d).

Step (e) may include adjusting a threshold mobility in the microchannel to fall between the mobilities of the free labeled substance and the probe substance bound to the label-free target substance. The label-free target substance of step (a) may use any one substance selected from a group consisting of a peptide, polypeptide, protein, ribonucleic acid (RNA), and deoxyribonucleic acid (DNA).

The probe substance of step (a) may use any substance selected from a group consisting of a peptide, polypeptide, protein, RNA, and DNA.

The protein may include CRISPR/dCas ribonucleoprotein (RNP). Specifically, the protein may include fluorescently labeled dead/deactivated Cas9 protein bound to single-guide RNA (sgRNA), crRNA, or tracrRNA.

The microchannel may include a main microchannel for receiving the sample solution and including a first electrode and a second electrode at both ends, and a buffer microchannel for receiving a buffer solution and including both ends connected to a third electrode or a ground voltage.

The electrical control conditions of step (e) may include at least one of a voltage and a current.

The mechanical control conditions of step (e) may include at least one of a pressure and a flow rate of the microchannel.

### Advantageous Effects

According to the afore-described embodiments of the present invention, the presence of a target substance may be determined based on the pattern and relative position of a fluorescence signal of a probe reagent for detection, without additional fluorescent labeling of the target substance.

Furthermore, label-free target deoxyribonucleic acid (DNA) may be rapidly detected with high sensitivity and resolution from a small amount of a sample.

In addition, because separation based on differences in mobility and concentration of substances occurs simultaneously due to the characteristics of the ion concentration polarization (ICP)-based selective preconcentration phenomenon, the sensitivity of optical detection may be improved.

Besides, the detection device design may be simplified by using a simple optical system with single-color fluorescence.

However, the scope of the present invention is not limited to the above effects.

### DESCRIPTION OF THE DRAWINGS

FIG. 1 is a flowchart of a concentration detection method for a label-free target, according to an embodiment of the present invention.

FIG. 2 is a detailed flowchart of a step of optimizing control conditions for concentration detection of a label-free target, according to an embodiment of the present invention.

FIG. 3 shows separated peaks of a preconcentration plug, according to an embodiment of the present invention.

FIG. 4 shows an example of a micro/nanofluidic concentration device where concentration detection of a label-free target is performed, according to an embodiment of the present invention.

FIG. 5 is a view for describing the formation and movement of an ion depletion zone and a preconcentration plug during concentration detection of a label-free target, according to an embodiment of the present invention.

FIG. 6 is a view specifically showing the movement of the preconcentration plug of FIG. 5.

FIG. 7 is an image showing the concentration pattern of a label-free target substance, according to a test example of the present invention.

FIG. 8 is an image showing the concentration pattern of a label-free target substance, according to another test example of the present invention.

### MODE OF THE INVENTION

Hereinafter, the present invention will be described in detail by explaining embodiments of the invention with reference to the attached drawings. The invention may, however, be embodied in many different forms and should not be construed as being limited to the embodiments set forth herein; rather, these embodiments are provided so that this disclosure will be thorough and complete, and will fully convey the concept of the invention to one of ordinary skill in the art. In the drawings, the thickness or size of layers are exaggerated for clarity and convenience of explanation.

As used herein, the term “probe” refers to a substance that specifically binds to a target substance to be detected in a sample, thereby enabling identification of the presence of the target substance in the sample through the binding. The probe substance may be a commonly used substance in the art and is not limited to a particular type. However, preferably, the probe substance may be a peptide, polypeptide, protein, ribonucleic acid (RNA), deoxyribonucleic acid (DNA), or microparticles including these substances.

As used herein, the term “target” refers to a substance, the presence of which in a sample is to be determined. The target substance may be a commonly used substance in the art and is not limited to a particular type. However, preferably, the target substance may be a peptide, polypeptide, protein, RNA, DNA, or microparticles including these substances.

More specifically, the target substance is a biomaterial derived from a living organism, a similar substance, or a substance produced outside a living organism, and refers to a substance including microparticles with micrometer- to nanometer-scale sizes. The target substance may be a substance derived from recombinant genes, recombinant proteins, cell lysates, blood, microalgae, or other fluids, but is not limited to these examples.

A concentration detection method for a label-free target, according to an embodiment of the present invention, will now be described.

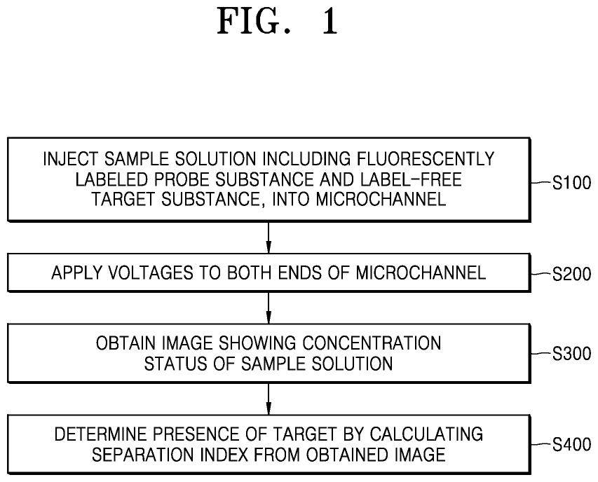

FIG. 1 is a flowchart of a concentration detection method for a label-free target, according to an embodiment of the present invention.

Referring to FIG. 1, a method of detecting a label-free target substance using ion concentration polarization (ICP) includes injecting a sample solution including a fluorescently labeled probe substance and a label-free target substance, into a microchannel (S100), applying a voltage to each end of the microchannel (S200), obtaining an image showing a concentration status of the sample solution (S300), and determining the presence of the target by calculating a separation index from the obtained image (S400).

Additionally, the method may further include controlling a preconcentration plug by adjusting electrical and/or mechanical control conditions to be applied to the microchannel (S500).

In general, not only the probe substance but also the target substance to be detected are fluorescently labeled in advance. However, labeling may alter the target substance or a low-molecular-weight substance may not be labeled. Furthermore, labeling consumes a large amount of the sample, requires two or three additional processes, and leads to significant quantitative errors due to variations in labeling efficiency among different types of proteins.

Thus, the present invention provides a method of controlling a preconcentration plug to enable the detection of a label-free target using only a fluorescence signal of a probe substance for detection.

Initially, a sample solution including a fluorescently labeled probe substance and a label-free target substance is injected into a microchannel (S100).

In step S100, the sample solution is injected into the microchannel, and no electric field is applied and no preconcentration plug is formed.

Then, a voltage is applied to each end of the microchannel (S200).

In step S200, an ion depletion zone is formed due to the ICP phenomenon in the microchannel, and an initial preconcentration plug observed as bright fluorescence is formed.

In this case, the applied voltage may be a voltage for implementing the ICP phenomenon. The initial voltage applied may be determined considering values such as the type of sample solution, the type of ion-selective membrane, a preset target plug position, and a flow rate of the sample solution in the microchannel.

Subsequently, an image showing a concentration status of the sample solution is obtained (S300).

In step S300, an image of a region where the sample is being concentrated in the microchannel is obtained in real time.

According to an embodiment, a region around the ion depletion zone where the preconcentration plug is formable may be observed using an optical device, and an image of the region around the ion depletion zone may be obtained at a preset cycle. For example, a video of a region where the sample is being concentrated in the microchannel may be obtained using an optical device such as a microscope, and a real-time image may be captured from this video to obtain the image of the region where the sample is being concentrated.

In step S300, when the fluorescent target is concentrated above a certain level, the position of the plug thereof may be identified in real time using an optical system. A video obtained using a camera of the optical system may be captured in real time to obtain the position and brightness values of each pixel, and the position and brightness information of the plug is calculated through appropriate filtering. To recognize the preconcentration plug separately from the background, a brightness value higher than that of the background is set as a threshold, and pixels with brightness values above the threshold are recognized as the preconcentration plug. In this case, various image processing techniques may be additionally applied to reduce noise and increase sensitivity.

Then, the presence of the target is determined by calculating a separation index from the obtained image (S400).

In step S400, the fluorescently labeled preconcentration plug remains as a single plug or splits into two or more plugs. Depending on the purity of the target substance, selective preconcentration into two or more plugs may occur.

For example, when the target substance to which the probe substance is to bind is not present, a single plug remains.

As another example, when the target substance is present in the sample, a portion of the probe substance successfully binds to the target substance, while the other portion remains unbound. Therefore, the labeled substances with different mobilities coexist in the micro/nanofluidic concentration device. Because the probe substance bound to the target substance and the probe substance remaining unbound coexist, separation into two or more plugs may occur and the presence of the target may be determined.

A case where a single preconcentration plug is separated into two plugs will now be described as an example.

For example, a single preconcentration plug may be separated into two plugs. One of the two plugs may be a preconcentration plug of a free labeled substance, i.e., the probe substance not bound to the target substance (hereinafter referred to as a first preconcentration plug), while the other may be a preconcentration plug of the probe substance bound to the target substance (hereinafter referred to as a second preconcentration plug).

The threshold mobility of a micro/nanofluidic system may be determined by the electrophoretic mobilities of cations and anions, diffusion coefficients, flow rate of the system, cross-sectional area, current, etc. A substance with a lower mobility than the threshold may be concentrated continuously at a point in the channel or with a concentration equilibrium point that moves relatively slowly. A substance with a higher mobility than the threshold mobility may be concentrated with a concentration equilibrium point that moves relatively rapidly.

That is, when two or more substances with different threshold mobilities coexist in a system, adjusting the threshold mobility to a value between the mobilities of the two substances may induce separation of preconcentration plugs based on different concentration patterns of the two substances.

For example, the first preconcentration plug may exhibit concentration with a concentration equilibrium point that moves, while the second preconcentration plug may exhibit concentration at a point in the channel or with a concentration equilibrium point that moves more slowly. Thus, two plugs are obtained.

In step S400, a separation index is calculated. The separation index may be calculated in real time based on the image obtained in real time in step S300.

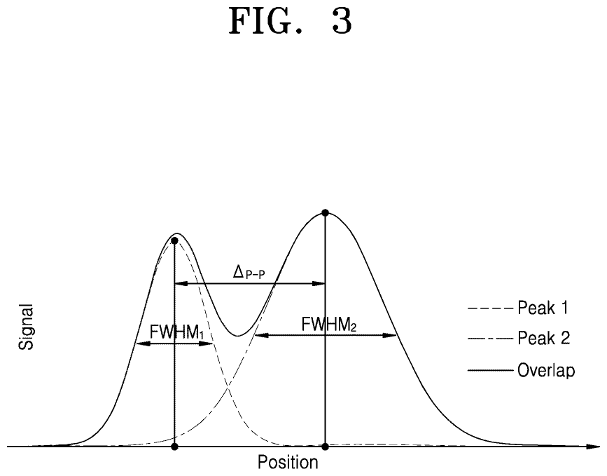

FIG. 3 shows separated peaks of a preconcentration plug, according to an embodiment of the present invention.

Referring to FIG. 3, when a preconcentration plug with a first peak Peak 1 and a second peak Peak 2 is observed, a separation index S may be obtained by calculating effective widths FWHM₁ and FWHM₂ of the preconcentration plug and a distance Δ_{P-P} between the peak centers.

Specifically, the separation index may be expressed by Equation 1.

$$Separation index ( S ) = \frac{{Δ}_{P - P}}{( {FWHM}_{1} + {FWHM}_{2} ) / 2} \qquad ( Equation 1 )$$

Although the present invention describes a preconcentration plug with two peaks, the same principle may be applied when three or more peaks are observed. For example, when a preconcentration plug with n peaks is observed, the separation index may be obtained by dividing the average distance between the n peak centers of the preconcentration plug by the average effective width of the peaks.

When the separation index exceeds a certain threshold, it indicates that two preconcentration plugs are distinguishable. The higher the separation index, the easier the first and second preconcentration plugs are distinguishable. On the other hand, when the target substance to which the probe substance is to bind is not present in the sample solution, a single plug remains and thus the separation index is calculated to be 0.

Meanwhile, in an embodiment of the present invention, after step S400, optimization may be performed to maximize a separation ratio of the preconcentration plug by real-time adjusting electrical or mechanical control conditions to be applied to the microchannel (S500).

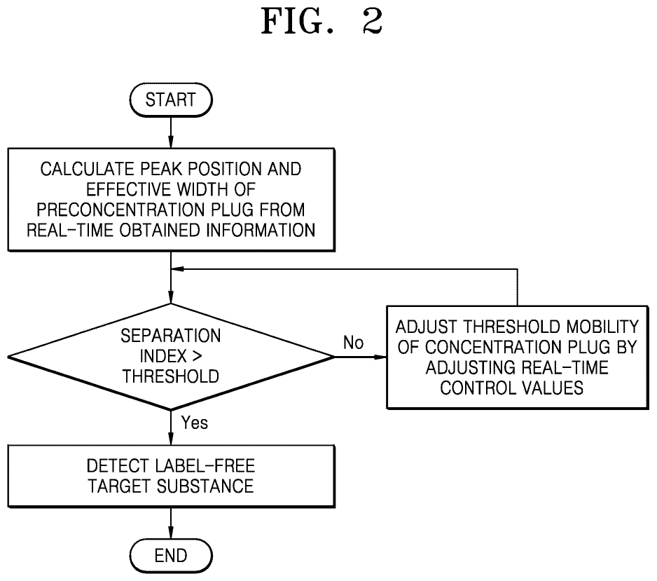

FIG. 2 is a detailed flowchart of the step of optimizing the control conditions, and a case where the target substance is present in the sample is assumed.

Referring to FIG. 2, a peak position and effective width of the preconcentration plug are calculated from the real-time image obtained in step S300. The peak position and effective width of the preconcentration plug are calculated from a region recognized as the preconcentration plug, i.e., pixels recognized as the preconcentration plug.

In this case, optionally, the electrical or mechanical control conditions to be applied to the microchannel may be adjusted in real time.

When the preconcentration plug is separated, the separation index is calculated. When the separation index is higher than a target threshold, a label-free target substance is detected. When the separation index is lower than the target threshold, optimization may be performed at least once to maximize a separation ratio of the preconcentration plug by real-time adjusting the electrical or mechanical control conditions to be applied to the microchannel. The optimization is to adjust the position of the preconcentration plug in the microchannel.

In an embodiment, the electrical control conditions changed to separate the preconcentration plug into two or more plugs may include the magnitude of a voltage or current applied to the microchannel. In another embodiment, the mechanical control conditions may include at least one of the pressure and flow rate of the microchannel.

In the step of optimizing the control conditions, to determine the concentration patterns of the two plugs, the threshold mobility in the microchannel may be adjusted to fall between the mobilities of the free labeled substance and the probe substance bound to the target substance.

Ultimately, when the calculated separation index is higher than the target threshold, the optimization of the control conditions is stopped and the label-free target substance is detected.

When the separation index is higher than a specific threshold, it indicates that preconcentration plugs are distinguishable, and thus the label-free target substance may be detected.

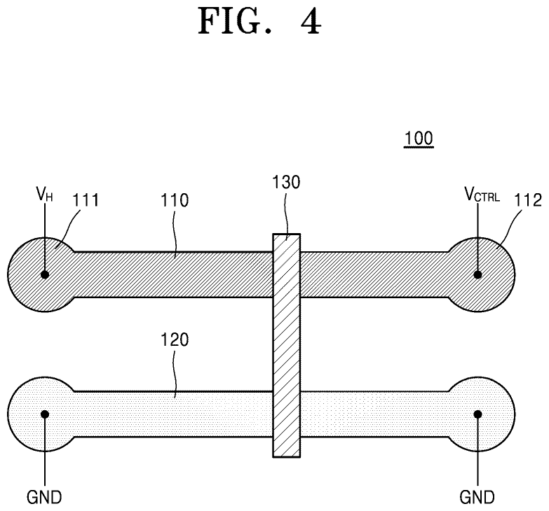

FIG. 4 shows an example of a micro/nanofluidic concentration device where concentration detection of a label-free target occurs, according to an embodiment of the present invention.

Referring to FIG. 4, a micro/nanofluidic concentration device 100 may include a main microchannel 110, a buffer microchannel 120, and an ion-selective membrane 130.

A sample solution may be injected into the main microchannel 110. The main microchannel 110 may include an inlet at an end to introduce the sample solution, and an outlet at another end to discharge the sample solution. In an embodiment, to facilitate the movement of the sample solution along its path, the main microchannel 110 may have a linear shape extending in one direction.

A reference voltage V_{H} may be applied to an end 111 of the main microchannel 110. The reference voltage V_{H} may be a fixed or variable voltage. A control voltage V_{CTRL}, which is changed by the control of a controller, may be applied to another end 112 of the main microchannel 110. The sample concentrator 100 may include electrodes capable of applying voltages to both ends 111 and 112 of the main microchannel 110.

A buffer solution may be injected into the buffer microchannel 120. In an embodiment, the buffer solution may be an aqueous electrolyte solution with a concentration corresponding to that of the substance injected into the main microchannel 110. The buffer microchannel 120 may have a “U” shape or a linear shape extending in one direction but is not limited to a specific shape.

Both ends of the buffer microchannel 120 may be connected to a ground voltage. The sample concentrator 100 may include electrodes capable of connecting both ends of the buffer microchannel 120 to the ground voltage. The buffer microchannel 120 may be used to increase the ion exchange efficiency through the ion-selective membrane.

In an embodiment, the microchannels 110 and 120 may have a width of 10 μm to 1000 μm and a height of 1 μm to 1000 μm. The microchannels 110 and 120 may include flexible polymer or hard plastic. For example, flexible polymer such as polydimethylsiloxane (PDMS), or hard plastic such as acrylic or polycarbonate may be used as the material of the microchannels. The microchannels 110 and 120 may have a linear or curved shape.

The ion-selective membrane 130 may be connected to each of the main microchannel 110 and the buffer microchannel 120 at one or more contact points. The ion-selective membrane 130 may induce the ICP phenomenon. The ion-selective membrane 130 may be a cation- or anion-selective membrane. In an embodiment, the ion-selective membrane 130 may be Nafion.

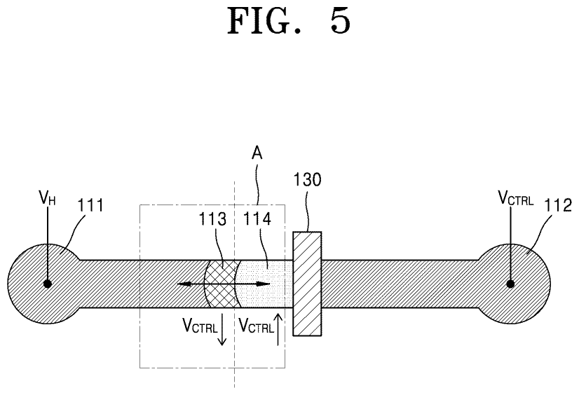

FIG. 5 is a view for describing the formation and movement of an ion depletion zone and a preconcentration plug in a micro/nanofluidic concentration device, according to an embodiment of the present invention.

Referring to FIG. 5, when electric fields V_{H} and V_{CTRL} are applied to the end 111 and the other end 112 of the main microchannel 110, the ICP phenomenon may occur near the ion-selective membrane 130 in the main microchannel 110, leading to the formation of an ion depletion zone 114. ICP is one of electrochemical transport phenomena observed around a structure with a nanomembrane. It is theoretically known that, when the thickness of an electric double layer is similar to the size of the nanomembrane, the overlapping of the electric double layer in the nanomembrane results in single-ion permeability. Ions with charges equal to wall charges are unable to pass through the nanomembrane due to diffusion and drift forces, and only ions with charges opposite to the wall charges pass through, leading to ion depletion and enrichment at the membrane interface.

Using the property that a sample solution with the same polarity as the nanomembrane may not pass through the ion depletion zone 114, the ion depletion zone 114 may be utilized as a mechanism for concentrating the sample solution. The sample solution may be concentrated starting from the boundary of the ion depletion zone 114 to form a preconcentration plug 113. A concentration equilibrium point of the sample solution may be determined based on advection caused by the flow and electro-migration caused by the electrophoretic mechanism. The concentration pattern of the sample solution may vary depending on whether advection or electro-migration is dominant.

The size of the ion depletion zone 114 may vary depending on the type of the ion-selective membrane 130, the potential difference between both ends 111 and 112 of the main microchannel 110, and the ion concentration distribution. For example, under specific initial conditions, when the potential difference between both ends 111 and 112 of the main microchannel 110 increases, that is, when the control voltage V_{CTRL} decreases, the area of the ion depletion zone 114 increases. As such, the preconcentration plug 113 at the boundary of the ion depletion zone 114 is pushed toward the end 111 of the main microchannel 110. On the other hand, when the potential difference decreases, that is, when the control voltage V_{CTRL} increases, the size of the ion depletion zone 114 decreases and the preconcentration plug moves toward the ion-selective membrane 130.

The size and position of the preconcentration plug 113 due to the ICP phenomenon may vary depending on the potential difference, current amount, flow rate, concentration time, channel shape, electrolyte type and concentration, etc. The method of detecting a label-free target substance, according to an embodiment of the present invention, may separate the preconcentration plug 113 and determine the presence of the label-free target substance, by real-time adjusting the factors capable of determining the size and position of the preconcentration plug 113.

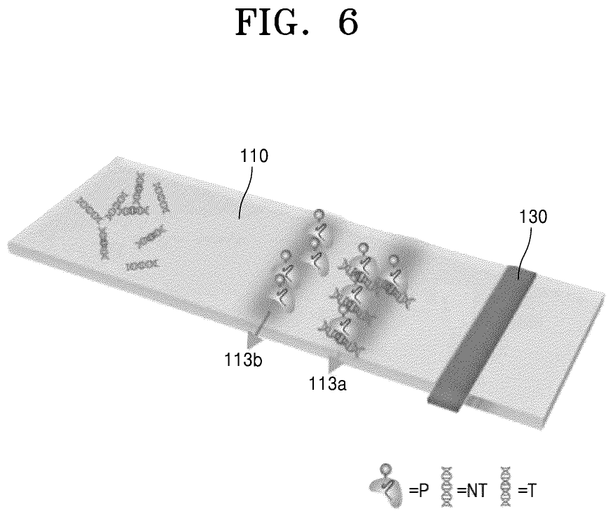

FIG. 6 is a view specifically showing the movement of a preconcentration plug in the micro/nanofluidic concentration device of FIG. 5.

Referring to FIG. 6, a target substance T and a non-target substance NT coexist in the sample solution, and a probe substance P, which specifically binds to the target substance T, is fluorescently labeled. When voltages are applied to the main microchannel 110, the ICP phenomenon occurs near the ion-selective membrane 130 in the main microchannel 110, forming a preconcentration plug.

The probe substance P bound to the target substance T (hereinafter referred to as a target-bound labeled substance) undergoes changes in molecular size and surface charge density. As such, its mobility in the micro/nanofluidic system changes. Specifically, the target-bound labeled substance has an absolute electrophoretic mobility lower than the threshold mobility in the micro/nanofluidic concentration device, and thus stacks at a specific point to form a first preconcentration plug 113a.

Meanwhile, the probe substance P not bound to the target substance T (hereinafter referred to as a free labeled substance) has an absolute electrophoretic mobility higher than the threshold mobility in the micro/nanofluidic concentration device. As such, a second preconcentration plug 113b may be formed by shifting the concentration equilibrium point, i.e., the position of the preconcentration plug, and propagate toward an end (e.g., toward the inlet or outlet).

As described above, the concentration pattern may be determined by comparing the absolute electrophoretic mobility of the sample solution with the threshold mobility. For example, fluorescent dCas9 protein, which is the probe substance P, exhibits differences in mobility depending on whether it binds to a nucleic acid, which is the target substance T. This property may be used to detect the presence of the target substance T in the sample solution based on the selective preconcentration pattern of the dCas9 protein.

As another example, a labeled nucleic acid with a specific sequence may be used as the probe substance P to determine and detect dCas9 protein or CRISPR/Cas system that binds to this sequence.

As an additional example, a labeled specific antigen or antibody may be used as the probe substance P to detect an antibody or antigen that binds thereto.

### Test Examples

Test examples will now be described for better understanding of the present invention. However, the following test examples are merely to promote understanding of the present invention, and the present invention is not limited to thereto.

### Embodiment 1

A micro/nanofluidic concentration device including a microchannel with a width of 150 μm and a height of 15 μm was prepared. A cation-selective membrane, Nafion, was used as the ion-selective membrane.

Epidermal growth factor receptor (EGFR) mutant DNA was used as the target substance, and dCas9 protein capable of binding to the EGFR mutant gene was used as the labeled substance. The labeled substance is attached with fluorescent mCherry molecules, enabling the observation of a preconcentration plug during fluorescence imaging.

As initial conditions, a reference voltage V_{H} of 60V was applied to an end of the main microchannel to form an initial preconcentration plug. When feedback control begins, a control value of V_{CTRL} is provided to another end of the main microchannel connected to a floating electrode.

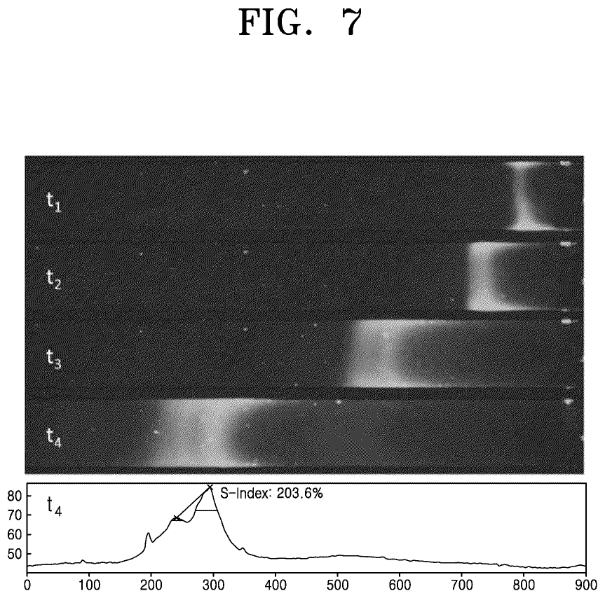

The results thereof are shown in FIG. 7.

Referring to FIG. 7, when an initial voltage is applied, concentration begins and a preconcentration plug starts to form. Simultaneously, the brightness of the middle region of the channel is analyzed in real time, and the peak position and effective width of the preconcentration plug are calculated.

By gradually lowering the current value to adjust the threshold mobility of the system during concentration, the difference in the movement speed of the preconcentration plug due to differences in mobility begins to increase.

The separation index at time point t₄ is calculated to be 203%, which indicates that two peaks are separated and distinguished from each other.

This demonstrates that two labeled substances with different mobilities are present in the sample. One of the two plugs is a preconcentration plug of the free labeled substance (i.e., dCas9), and the other is a preconcentration plug of the labeled substance bound to the target substance (i.e., EGFR mutant DNA-dCas9 complex).

### Embodiment 2

The test was conducted under the same conditions as Embodiment 1, except that EGFR wild-type DNA was used as the target substance.

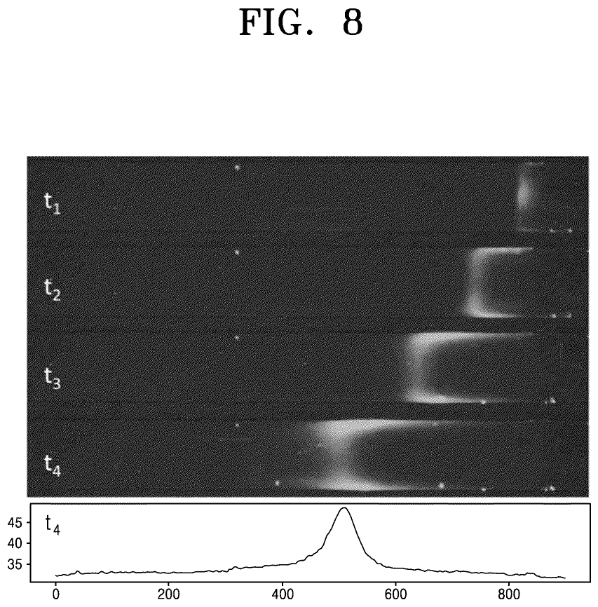

The results thereof are shown in FIG. 8.

Referring to FIG. 8, when an initial voltage is applied, a single preconcentration plug begins to form as in the positive sample.

However, even when the current value is gradually lowered to adjust the threshold mobility of the system during concentration in the same manner as Embodiment 1, the single preconcentration plug is not separated but moves away from the ion-selective membrane during concentration.

This indicates that only one type of labeled substance, i.e., free dCas9 protein, is present in the micro/nanofluidic concentration device.

Therefore, the presence of label-free EGFR mutant DNA may be determined based on the selective preconcentration results obtained using labeled EGFR mutant DNA-specific dCas9 protein.

According to the afore-described embodiments of the present invention, a detection method capable of improving detection sensitivity by enabling substance concentration based on the ICP phenomenon may be provided.

Furthermore, according to an embodiment of the present invention, using a micro/nanofluidics-based device, detection may be enabled from a small amount of sample without an additional labeling process, and precise detection may be achieved by controlling fluidic and electrical conditions in real time.

Additionally, according to an embodiment of the present invention, a simple optical system using single-color fluorescence may be employed and thus the device design required for detection may be simplified.

The afore-described embodiments of the present invention are applicable to various fields including ICP-based diagnosis, performance testing of modified proteins, and water quality testing.

While the present invention has been particularly shown and described with reference to embodiments thereof, it will be understood by one of ordinary skill in the art that various changes in form and details may be made therein without departing from the scope of the present invention as defined by the following claims.

1. A method of detecting a label-free target substance using ion concentration polarization (ICP), the method comprising:
(a) injecting a sample solution comprising a fluorescently labeled probe substance and a label-free target substance, into a microchannel;
(b) applying a voltage to each end of the microchannel;
(c) obtaining an image showing a concentration status of the sample solution; and
(d) determining presence of the target by calculating a separation index from the obtained image.

2. The method of claim 1, wherein, in step (b), an ion depletion zone and a preconcentration plug are formed.

3. The method of claim 1, wherein, in step (d), a single preconcentration plug is separated into two plugs, and
wherein one of the two plugs is a preconcentration plug of a free labeled substance (hereinafter referred to as a first preconcentration plug), while the other is a preconcentration plug of a labeled substance bound to the label-free target substance (hereinafter referred to as a second preconcentration plug).

4. The method of claim 3, wherein the first preconcentration plug exhibits concentration with a concentration equilibrium point that moves, while the second preconcentration plug exhibits concentration at a point in the channel or with a concentration equilibrium point that moves more slowly than the first preconcentration plug.

5. The method of claim 3, wherein the separation index is calculated by Equation 1:
$$Separation index ( S ) = \frac{{Δ}_{P - P}}{( {FWHM}_{1} + {FWHM}_{2} ) / 2}$$
wherein Δ_{P-P} denotes a distance between centers of the first and second preconcentration plugs, FWHM₁ denotes an effective width of the first preconcentration plug, and FWHM₂ denotes an effective width of the second preconcentration plug.

6. The method of claim 1, wherein, when the separation index is lower than a target threshold in step (d), (e) adjusting electrical or mechanical control conditions to be applied to the microchannel is performed at least once after step (d).

7. The method of claim 6, wherein step (e) comprises adjusting a threshold mobility in the microchannel to fall between mobilities of the free labeled substance and the probe substance bound to the label-free target substance.

8. The method of claim 1, wherein the label-free target substance of step (a) uses any one substance selected from a group consisting of a peptide, polypeptide, protein, ribonucleic acid (RNA), and deoxyribonucleic acid (DNA).

9. The method of claim 1, wherein the probe substance of step (a) uses any one substance selected from a group consisting of a peptide, polypeptide, protein, RNA, and DNA.

10. The method of claim 8, wherein the protein comprises CRISPR/dCas ribonucleoprotein (RNP).

11. The method of claim 1, wherein the microchannel comprises:
a main microchannel for receiving the sample solution and comprising a first electrode and a second electrode at both ends; and
a buffer microchannel for receiving a buffer solution and comprising both ends connected to a third electrode or a ground voltage.

12. The method of claim 6, wherein the electrical control conditions of step (e) comprise at least one of a voltage and a current.

13. The method of claim 6, wherein the mechanical control conditions of step (e) comprise at least one of a pressure and a flow rate of the microchannel.

14. The method of claim 9, wherein the protein comprises CRISPR/dCas ribonucleoprotein (RNP).
